(19) United States
(12) Patent Application Publication
(10) Pub. No.: US 2004/0147789 A1
(43) Pub. Date: Jul. 29, 2004

(54) Allyl esters substituted by a difluoromethylene group, their process of synthesis and their use, and a process for functionalizing a double bond

(75) Inventor: Nicolas Roques, Gaillac (FR)

Correspondence Address: BURNS DOANE SWECKER & MATHIS L L P, POST OFFICE BOX 1404, ALEXANDRIA, VA, 22313-1404, US

(73) Assignee: RHODIA CHIMIE, Boulogne Billanocurt Cedex (FR)

(21) Appl. No.: 10/740,802

(22) Filed: Dec. 22, 2003

Related U.S. Application Data
(63) Continuation-in-part of application No. 10/203,075, filed on Aug. 6, 2002.
Application No. 10/203,075 is a 371 of international application No. PCT/FR01/00364, filed on Feb. 12, 2001.

(30) Foreign Application Priority Data
Feb. 11, 2000 (FR) 00/01744
Dec. 20, 2002 (FR) 02/16308

(51) Int. Cl. C07C 43/02

(52) U.S. Cl. 568/662

(57) ABSTRACT

This invention provides compounds of formula:

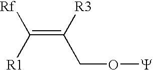

in which R_{f} is a radical that carries a perfluoromethylene group, which group ensures bonding to the remainder of the molecule; R1 and R3, which can be the same or different, are chosen from among hydrogen and alkyl or aryl radicals; Ψ is an electroattractor group such that Ψ-O—H is an acid whose pKa (in water) is at most equal to 8, advantageously 6, and preferably 5. These compounds are useful for the synthesis of nitrogen-containing heterocyclic compounds.

### CROSS-REFERENCE TO RELATED APPLICATIONS

This application is a continuation-in-part of U.S. Ser. No. 10/203,075, filed Aug. 6, 2002 (Attorney Docket No. RN00011); which application is a 371 of International Application No. PCT/FR01/00364 filed on Feb. 12, 2001, which application claims the benefit of French Application 00/01744 filed on Feb. 11, 2000. This application also claims benefit of French Application 02/16308 filed on Dec. 20, 2002. Each of these applications are incorporated herein by reference in their entirety.

### FIELD OF INVENTION

The present invention relates to allyl esters substituted by a difluoromethylene group, their process of synthesis and their use. The invention also relates to processes for functionalizing a double bond. This invention has as its object compounds that form an allyl ester that is substituted by a difluoromethylene group. It also has as its object a method of synthesis of these compounds as well as their use in cycloaddition processes, specifically 3+2 cycloadditions.

### BACKGROUND

Compounds comprising perhalogenated and especially perfluoro carbon atoms play an increasingly important role in derivatives with biological activity, whether this biological activity is exerted in the animal kingdom or in the plant kingdom.

The synthesis or grafting of these perhalogenated and more preferentially perfluoro compounds is often difficult and requires expensive means.

During the last decade, compounds that carry fluoroalkyl groups have become very widespread, especially in agrochemistry and pharmaceutical products.

In particular, heterocyclic compounds, specifically those containing nitrogen and especially with 5 links, have become relatively common when they carry (—CF₂) groups. Nevertheless, synthesis of such compounds is difficult and often requires an elevated sequence of stages.

Among the techniques that have been proposed is the one consisting in subjecting a double bond to the action of a sulfonyl halide. In particular, the action of trifluoromethanesulfonyl bromide has already been described in the Bulletin de la Societe Chimique de France, No. 6, 1986, from page 881 to page 884.

However, sulfonyl bromide is difficult and expensive to handle, especially on account of the high instability of this bromide. In addition, the bromide, like the chloride, is a powerful oxidizing agent which can modify the capacities for survival of the products obtained during this addition. The bromide ions formed during the reaction that react with the residual bromide can lead to bromine, which is then a source of further spurious reactions.

Trifluoromethanesulfonyl chloride, which is occasionally known as triflyl chloride, is significantly less reactive than the bromide; thus, it has been attempted to use very specific ruthenium complexes (Ru(PPh₃)Cl₂) to catalyze the addition to double bonds. This addition appears to be relatively versatile, but the reactions are often difficult to reproduce and the results appear to be somewhat erratic.

It has also been proposed, in the case of double bonds bearing a silicon atom, to use free-radical generators such as tert-butyl peroxide. One of the major risks of this technique lies in the fact that double bonds, under the action of free radicals, have a tendency to polymerize and not to lead to the addition reaction. Another risk is an oxidation with a chlorine according to a mechanism of positive chlorine type.

### SUMMARY

The present invention relates to a process for functionalizing a double bond, and more particularly a double bond bearing a metalloid atom. More particularly, the present invention is directed toward adding to a double bond, on the one hand, and to one of the atoms a halogen atom and, on the other hand, to the other carbon-based radical whose carbon atom is perhalogenated. Said functionalization is produced by the action of perhalogenated sulphonyl chloride on the carbon bearing sulphur in the presence of a free radical initiator, preferably by homolytic cleavage. The invention is useful in organic synthesis.

One embodiment relates to a process for functionalizing a double bond corresponding to the following formula I:

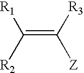

wherein:

R₁, R₂ and R₃, which are identical or different, are hydrogen atoms or hydrocarbyl groups attached to said double bond via an sp³ carbon,

Z is selected from the group consisting of the following:

halogen atoms,

residues such that ZH is an oxygenated acid,

groups of formula (CHR′)ₘ- [symbol] , wherein [symbol] is an halogen atom, and

groups of formula (CHR′)ₘ—Y(O)_{q}—R₄, wherein:

—Y is a chalcogen,

q is zero or an integer of not more than 3, with the condition that when Y is oxygen, q is equal to zero,

R′ represents a hydrocarbyl group, or Hydrogen atom,

m is equal to 1 or zero, and

R₄ is a hydrocarbyl or silyl group,

said process comprising the step reacting said double bond with a sulfonyl chloride compound perhalogenated on a sulfur-bearing carbon of the sulfonyl function, in the presence of a free-radical initiator.

In another embodiment, the invention relates to a process according wherein the free-radical initiator is an initiator by homolytic cleavage. Z may be selected from the group consisting of the following:

chlorine atom, fluorine atom, and

groups of formula (CHR′)ₘ—Y(O)_{q}—R₄, wherein:

Y is a light chalcogen,

q is zero or an integer of not more than 1, with the condition that when Y is oxygen, q is equal to zero,

R′ represents a hydrocarbyl group of not more than four carbons, or a hydrogen atom,

m is equal to zero, and

R₄ is a hydrocarxbyl or silyl group.

In a preferred embodiment, has the formula Y—R₄ wherein Y is a light chalcogen, R₄ is an electron-withdrawing group and/or the perhalogenated sulfonyl chloride has the formula R_{f}—SO₂—Cl, wherein R_{f} has the formula

EWG-(CX₂)ₚ—, wherein:

X groups, which are similar or different, represent a chlorine atom, a fluorine atom or a group of formula CₙF₂ₙ₊₁ wherein n is an integer of not more than 5, with the condition that at least one of the X groups is fluorine,

p is integer of not more than 2,

EWG is an electron-withdrawing group, optionally comprising functions, said functions being inert under the reaction conditions, and

the total number of carbon atoms in R_{f} is of between 1 and 15.

In yet another embodiment, the X groups, which are similar or different, represent a chlorine atom, a fluorine atom or a group of formula CₙF₂ₙ₊₁ wherein n is an integer of not more than 2, with the condition that at least one of the X groups is fluorine, EWG is an electron-withdrawing group, comprising inert functions under the reaction conditions, said functions being a fluorine atom or a perfluoro residue of formula CₙF₂ₙ₊₁, with n being an integer of not more than 8, the total number of carbon in R_{f} is of between 1 and 10.

In an embodiment, R_{f} contains not more than 6 carbon atoms, preferably not more than 3 carbon atoms. In an embodiment, Y is oxygen, R₄ is an acyl group comprising not more than 15 carbon atoms, preferably not more than 10 carbon atoms.

In a preferred embodiment, the invention also relates to a compound of the following formula (II), comprising a functionalized double bond,

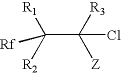

wherein R₁, R₂, R₃, Z and R_{f} are as defined for formula (I) above, with the following additional conditions:

at least one of the groups R₁ or R₂ is Hydrogen atom,

Z is a group of formula (CHR′)ₘ—Y—R₄ wherein R₄Y is such that R₄YH is an oxygenated acid,

the compound of formula (II) optionally comprises an aromatic nucleus, said aromatic nucleus being separated from the said functionalized double bond by at least two atoms of sp³ hybridization, in the case of Z, an oxygen atom and at least one carbon atom, and in the other cases, at least two sp³ carbon atoms, and

the total number of carbon atoms in the compound is of at least equal to (6-m) and not more than 30.

In the above formula, both groups R₁ or R₂ may be hydrogen.

In yet another embodiment, the invention relates to a process for preparing 3-perfluoroalkyl-1,2-epoxypropane, comprising the use of the compound of formula (II) defined in claim 23, wherein m is equal to 1.

An embodiment also relates to a compound of the following formula (III)

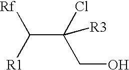

wherein R_{f}, R₁ and R₃ are as defined above. The above compound can also be used in a process for preparing 3-perfluoroalkyl-1,2-epoxypropane.

The invention also relates to a compound of formula (I)

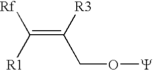

in which

R_{f} is a radical that carries a perfluoromethylene group, which group ensures bonding to the remainder of the molecule;

R1 and R3, which can be the same or different, are chosen from among hydrogen and alkyl or aryl radicals; and

Ψ is an electroattractor group such that Ψ-O—H is an acid whose pKa (in water) is at most equal to 8, advantageously 6, and preferably 5. Ψ may be an electroattractor group such that Ψ-O—H is an acid whose pKa (in water) is at least equal to 1, advantageously 2. Preferably, at least one of R1 and R3 is a light alkyl (at most 4 carbons), or a hydrogen, advantageously a hydrogen, and/or the R_{f} radical has the following formula (II):

GEA-(CX₂)ₚ—

where

X, the same or different, represent a chlorine, a fluorine, or a radical of formula CₙF₂ₙ₊₁ with n an integer that is at most equal to 5, preferably 2, with the condition that the X of the methylene group that carry the open bond are not chlorine and that one of them at least is a fluorine;

GEA is a hydrocarbon group or an electroattractor group (i.e., the Hammett constant cap (sigma p) is >0, advantageously at least equal to 0.2, preferably inert, advantageously when p is equal to 1, an electroattractor group; and

p is a positive integer.

In other preferred embodiments, R1 is hydrogen, R3 is hydrogen and/or R_{f} is a perfluoroalkyl of general formula CᵣF₂ₙ₊₁, where r is a positive integer from 1 to 10, advantageously from 1 to 5, preferably from 1 to 3. Preferably, both R1 and R3 are hydrogen. Preferably, R_{f} is chosen from among the trifluoromethyl, pentafluoroethyl and heptafluoropropyl radicals.

In other preferred embodiments, Ψ is an acyl, preferably an acyl such that the pKa of Ψ-O—H is at least equal to roughly 2, advantageously Ψ-O—H is an alkanoic acid with 1 to 8 carbon atoms, preferably from 2 to 5.

The invention also relates to a process of synthesis of the compound of formula (1), wherein it entails bringing into contact a compound of formula (III):

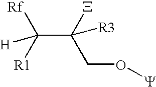

where [symbol] is a starting group that is chosen from among pseudohalogens and halogens, advantageously chlorine or bromine; with a base that is chosen from among strong nitrogen-containing bases, of which the associated acid has a pKa that is at least equal to 12 and/or among anionic bases with the condition that when the base is a non-nitrogen-containing anionic base, the latter is in the presence of a solvent or a mixture of polar solvents. The base may be a non-nitrogen-containing anionic base and wherein said polar solvent has a solvent whose donor index is at least equal to 10, advantageously 15, and preferably 20. The base may be a non-nitrogen-containing anionic base and wherein said polar solvent is a water-miscible solvent in any proportion. The base may be a non-nitrogen-containing anionic base and wherein said polar solvent does not have an acid function, i.e., that the pKa of the most acidic hydrogen of said solvent is at least equal to 20, advantageously 25, and preferably 30. The base is a non-nitrogen-containing anionic base, advantageously chosen from among salts, especially alkaline or alkaline-earth salts, of silylated amines and silyl amines. The base may be the anion of a silylamine chosen from among alkaline and alkaline-earth salts of HMDZ (hexamethyldisilazane). The base may be used in the presence of a polar, advantageously aprotic, solvent. The base may also be a base that carries at least 2 trivalent nitrogens, preferably such that said 2 nitrogens are conjugated via at least one double bond, more preferably such that said 2 trivalent nitrogens form a bond system comprising an imine conjugated with the doublet of an amine.

The compound of formula (I) may be used as a precursor of a heterocyclic compound substituted by a group R_{f} by cyclocondensation with a co-substrate that carries 2 double bonds. Preferably, the cyclocondensation is of type 3+2.

Preferably, the co-substrate is an organic compound that carries a pentavalent nitrogen, that itself carries 2 double bonds (including donor-acceptor type bonds), of which at least one double bond links said nitrogen to a carbon.

### DETAILED DESCRIPTION OF PREFERRED EMBODIMENTS

In the course of the study that led to the present invention, it has been possible to show that the action of sulfonyl chlorides on double bonds activated with an aromatic nucleus leads essentially to polymerization reactions rather than addition reactions.

In addition, certain double bonds, especially those that present major advantages for manufacturing synthons, are often not reactive enough to form the desired addition compounds.

It is thus seen that the decomposition of sulfonyl chlorides that are perhalogenated on the sulfur-bearing carbon of the sulfonyl bonds does not give an addition reaction on a double bond with good results, except in an extremely limited number of already-functionalized products.

Accordingly, one of the aims of the present invention is to define a family of compounds bearing already-functionalized double bonds, that can give acceptable addition yields with a technique of decomposition of sulfonyl chloride initiated by free radicals.

Another aim of the present invention is to provide an optimization of the operating conditions for this novel family of compounds.

Another aim of the present invention is to provide a process that does not require expensive catalysts such as those based on metals from column VIII, especially of the platinum mine, and in particular ruthenium.

Another aim of the present invention is to provide a process in which ruthenium, especially in the form coordinated with phosphines and especially aromatic phosphines, is present in an amount such that the [Ru]/[sulfonyl chloride perhalogenated on the sulfur-bearing carbon] molar ratio is not more than 1‰, advantageously 0.1‰ and preferably 0.01‰. It is even preferable for it not to be present.

Another aim of the present invention is to provide a process in which the platinum mine metals are present in an amount such that the [sum of the platinum mine metals]/[sulfonyl chloride perhalogenated on the sulfur-bearing carbon] molar ratio is not more than 1‰, advantageously 0.1‰ and preferably 0.01‰. It is even preferable that they are not present.

Another aim of the present invention is to provide a process in which the metals from column VIII are present in an amount such that the [sum of the metals from column VIII]/[sulfonyl chloride perhalogenated on the sulfur-bearing carbon] molar ratio is not more than 1‰, advantageously 0.1‰ and preferably 0.01‰. It is even preferable for them not to be present.

Another aim of the present invention is to provide novel intermediates allowing novel synthetic routes.

The Periodic Table of the Elements used in the present patent application is that of the supplement to the Bulletin de la Société Chimique de France, January 1966, No. 1.

It may be useful to recall that the expression “identical or different” is used to indicate that the substituents under consideration may or may not be the same.

These aims and others which will become apparent hereinbelow are achieved by means of a process for functionalizing a double bond corresponding to formula I:

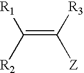

in which R₁, R₂ and R₃, which may be identical or different, are chosen from hydrogen and hydrocarbyls attached to said double bond via a carbon of sp³ hybridization; in which Z is chosen from:

halogens, advantageously chlorine and fluorine;

residues such that ZH is an oxygenated acid;

radicals of formula (CHR′)ₘ- [symbol] with [symbol] chosen from halogens;

and radicals of formula (CHR′)ₘ—Y(O)_{q}—R₄ in which

Y is a chalcogen, advantageously a light chalcogen;

q is zero or an integer not more than 3, advantageously not more than 2 and preferably not more than 1, with the condition that when Y is oxygen, q is equal to zero;

R′ represents a hydrocarbyl, advantageously of not more than four carbons, or preferably a hydrogen;

m is equal to 1 or preferably to zero;

and R₄ is chosen from a hydrocarbyl or silyl group;

by the action of sulfonyl chloride perhalogenated on the sulfur-bearing carbon (of the sulfonyl function) in the presence of a free-radical initiator, preferably by homolytic cleavage.

The expression “perhalogenated carbon” should be understood as meaning a carbon of sp³ nature optionally substituted with not more than two, and advantageously not more than one, electron-withdrawing group(s), and bearing no hydrogen, all the other atoms being halogens. It is preferable for these halogens borne by said perhalogenated carbon all to be chlorine or fluorine and preferably all fluorine.

It is important that the radicals R₁, R₃ and R₂, when they are hydrocarbyls (i.e. comprising hydrogen and carbon, but possibly comprising other atoms), are attached to the double bond via one of their sp³ carbons, otherwise the reactivity toward the sulfonyl chloride is greatly affected thereby. In particular, the presence of an aromatic directly attached to the double bond plays an extremely unfavorable role. It is also recommended to avoid carbon atoms that are both allylic and benzylic. More generally, even when they are not conjugated with said double bond, the presence of an aromatic in the molecule is unfavorable. Thus, the presence of an aromatic in Z, especially when ZH is an oxygenated acid, is unfavorable especially when an aromatic nucleus is separated from a carbon of said double bond by less than two atoms of sp³ hybridization (oxygen or carbon, preferably at least two sp³ carbons).

It should be noted that there is overlap between the definition of Z, Z=(CHR′)ₘ—Y(O)_{q}—R₄ and that which indicates that Z is such that ZH is an oxygenated acid, especially when m is equal to zero, when q is equal to zero, when Y is oxygen and R₄ is a hydrocarbon radical attached to Y via an electron-withdrawing function such as, especially, carbonyl (—CO—), carbonate (—CO—O—), sulfonyl (SO₂), sulfoxide (—SO—), sulfate (—SO₃—), phosphate [P(═O)(—O—)O—], phosphonate [P(═O)(—O—)—] and phosphinate [P(═O)(−)-].

For reasons of steric bulk, it is recommended to choose the radicals R₁, R₃ and R₂ such that at least one and preferably two of these three radicals is hydrogen. It is also preferable that neither R₁ nor R₂ is tertiary. It is also preferable that at least one, and even both, of the radicals R₁ and R₂ are hydrogen.

It is preferable that the initiator generating the free radicals releases these radicals after a homolytic cleavage, i.e. a cleavage taking place between two atoms of the same element and generating an electron radical on each of the two atoms. This cleavage may be actinic, catalytic or, preferably, thermal.

The cleavage may also be heterolytic when systems are used involving metals that have two valency states (iron or copper) with peroxides and especially hydroperoxides.

Examples of preferred initiators that should be mentioned include various peroxides, preferably symmetrical, and various azo compounds, such as azobisisobutyronitrile (reference may be made especially to the “Polymer Handbook”). Among the peroxides, mention may be made of alkyl peroxides and especially tert-alkyl peroxides, and acyl peroxides, especially alkanoyl peroxides, that are preferably symmetrical.

The acyl peroxides that may be used are preferably peroxides whose acyls are of low molecular weight, i.e. their carbon number is not more than 10, and preferably not more than 6 when they are aliphatic, but it is preferable to use acyl peroxides of aromatic nature, for instance benzoyl peroxide.

The free-radical initiator is advantageously not more than 0.2 times the molar amount of the sulfonyl chloride, preferably not more than 0.1 times, the optimum zone being between 1% and 8% of the amount of sulfonyl chloride. The reaction is advantageously conducted such that the release of the free radicals takes place gradually. A good technique for achieving this objective consists in adding the initiator, i.e. the free-radical generator, slowly and gradually. Another technique consists in using a temperature that allows the release of the free radicals to be controlled.

The reaction temperature is regulated such that it is between ambient temperature and 150° C., preferably between 50 and 120° C. and more preferentially between 60 and 100° C.

It should be noted that, for certain substrates, the reaction does not need an initiator, especially for substrates whose double bond bears a chalcogen, usually an oxygen.

However, the use of a free-radical generator still improves the reaction yield.

The amount of substrate relative to the sulfonyl chloride is about once the molar amount. However, to satisfy specific problems, it is possible to depart considerably from this value which corresponds to the stoichiometric value as defined by the following reaction:

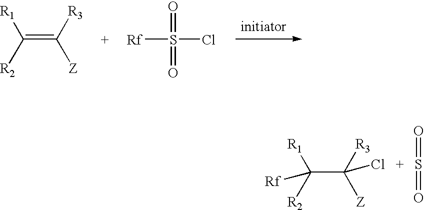

In particular, when there is a large risk of polymerization, a smaller amount of compounds bearing a double bond may be used relative to the amount of sulfonyl chloride. Thus, it is possible to vary very widely the ratio between the substrate bearing the double bond and the sulfonyl chloride. However, in the majority of cases, the ratio is of the order of 1±0.5.

The pressure may vary within large proportions but it is preferable to work at an autogenous pressure or at atmospheric pressure.

Although its presence is not necessary, it is possible to use a solvent. The solvents that may be used are solvents that are inert toward sulfonyl chloride and that do not constitute free-radical traps.

Mention may thus be made of the hydrocarbons and halogenated derivatives that are usually used in free-radical chlorination reactions.

More specifically, alkanes with a suitable boiling point, i.e. whose boiling point is at least equal to the temperature at which it is desired to work; petroleum fractions; aromatic chloro derivatives, are all entirely acceptable.

The sulfonyl chlorides that are preferred are those corresponding to the formula R_{f}—SO₂—Cl in which R_{f} corresponds to formula (IV):

EWG-(CX₂)ₚ—

in which:

the radicals X, which may be similar (i.e. they are identical) or different, represent a chlorine, a fluorine or a radical of formula CₙF₂ₙ₊₁ with n being an integer not more than 5 and preferably not more than 2, with the condition that at least one of the radicals X is fluorine; when p is equal to 1,

EWG is an electron-withdrawing group (that is to say that the Hammett constant sigma p is >0, advantageously at least equal to 0.2), otherwise it may be any radical, which is preferably inert, and advantageously an electron-withdrawing group (cf. preceding lines);

p is a positive integer, i.e. it cannot comprise the value 0.

EWG is advantageously fluorine, especially when p is less than or equal to 2.

The radicals X are advantageously all fluorine, especially when p is less than or equal to 2.

Another value of EWG (electron-withdrawing group) is when it equals chlorine; in this case, EWG is a chlorine.

p represents an integer advantageously not more than 4 and preferably not more than 2;

EWG advantageously represents an electron-withdrawing group whose optional functions are inert under the reaction conditions, advantageously fluorine or a perfluoro residue of formula CₙF₂ₙ₊₁, with n being an integer not more than 8 and advantageously not more than 5.

The total carbon number of Rf is advantageously between 1 and 15 and preferably between 1 and 10.

It is advantageous that Rf should be of formula CᵣF₂ᵣ₊₁ with r being an integer not more than 15 and advantageously between 1 and 10.

The present invention is particularly advantageous for radicals R_{f} of low molecular weight, i.e. those that are relatively volatile (with a boiling point at atmospheric pressure of not more than 100° C). The technique is particularly advantageous for radicals R_{f} containing a radical with an odd number of carbons, and particular mention should be made for radicals R_{f} of C₁, C₂ and C₃.

Radicals R_{f} higher than C₆ are less advantageous.

The compounds that give the most advantageous results are those in which Z is of structure Y—R₄ with Y being a chalcogen, preferably a light chalcogen, i.e. sulfur or oxygen and more particularly the latter.

R₄ is a hydrocarbyl group, i.e. a group containing hydrogen and carbon. These hydrocarbyl groups may be alkyl (i.e. an alcohol residue of which the hydroxyl function is ignored), an aryl or an oxygenated acid residue (i.e. a residue whose acidic hydrogen is borne by an oxygen) from which an OH function has been removed. In this latter group, mention may be made of acyls, phosphoryls, phosphonyls, phosphinyls and sulfonyls. The acids R₄Y(O)_{q}— advantageously have a pKa of not more than 10, preferably not more than 8 and more preferentially not more than 6. Acids with a Hammett constant that is greater than or equal to that of perfluoroalkanesulfonic acids, and especially triflic acid, are not preferred.

The group R₄ is advantageously an electron-withdrawing group of the acyl type.

It is preferable for the molecule bearing the double bond not to contain a strongly reductive function, or a nucleophilic function capable of reacting with the sulfonyl chloride.

The total carbon number of the substrate of formula I is generally not more than 50 (one significant figure) and better still not more than 30. In particular, when R_{f} is of the type CX₃ (CX₂)ₛ, the presence of halogens heavier than fluorine at the other end of the chain Rf liable to give spurious reactions, especially when the chain Rf is short (s less than or equal to 5 and, even, less than or equal to 4). As has been stated previously, another aim of the present invention is that of providing reaction intermediates that allow novel routes of access.

This aim is achieved by means of compounds corresponding to the formula:

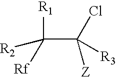

with R₁, R₂, Z and Rf being chosen from the same values (and with the same preferences) as above, but with the following additional conditions:

with R₁ and R₂ chosen from hydrogen and hydrocarbyl radicals, with the condition that one of the radicals R₁ or R₂ at least is equal to H, and advantageously both of them;

with Z chosen from radicals of formula (CHR′)ₘ—Y—R₄ in which R₄—Y is such that R₄YH is an oxygenated acid,

the possible aromatic nucleus (nuclei) being separated from said double bond by at least two atoms of sp³ hybridization (in the case of Z, an oxygen atom and at least one carbon atom, advantageously at least two sp³ carbon atoms; in the other cases, at least two sp³ carbon atoms); the total carbon number of the molecule being at least equal to (6-m) and not more than 30, and advantageously in which:

Y is a chalcogen, advantageously a light chalcogen, preferably oxygen;

R′ represents a hydrocarbyl, advantageously of not more than four carbons, or preferably a hydrogen;

m is equal to 1 or, preferably, to zero;

and R₄ is chosen from hydrocarbyl groups, advantageously from acyls.

It is advantageous for R_{f} to be of formula CᵣF₂ᵣ₊₁ with r being an integer not more than 15, advantageously between 1 and 10 and preferably not more than 4.

For steric reasons, it is preferable for said acid R₄—YH not to comprise any branching alpha or beta to the atom bearing the acidic proton, in general oxygen; thus, for example, in the case of a carboxylic acid, the atom bearing the carboxylic function, which is beta to the oxygen bearing the proton, is advantageously neither tertiary nor even secondary, nor does it correspond to the branching of an aromatic nucleus.

One subfamily of the above compounds is particularly novel, namely the family in which m is equal to zero; when R₃ is other than H, the compounds are highly reactive [lacuna] constitutes only reaction intermediates but remains identifiable, especially at low temperature by fluorine-19 NMR. When R₃ is hydrogen, these compounds are surprisingly stable. These two subfamilies decompose or are hydrolyzed to carbonyl, aldehyde or ketone derivatives, see the examples. This subfamily may thus be used to synthesize by hydrolysis, for example acid hydrolysis, aldehydes or ketones and derivatives thereof. A subsequent oxidation, which is known per se, of the aldehydes gives the corresponding acids.

Another advantageous subfamily lies in the alcohol and the corresponding esters in which m is equal to one and Y is oxygen and in which, advantageously, R₃ is H.

The alcohol is readily synthesized by alcoholysis of the corresponding ester, without touching the chlorine borne by the carbon atom adjacent to that bearing the ester function and then the alcohol:

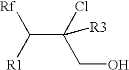

The alcohol or ester readily lead, especially under the action of strong base (sodium hydroxide, potassium hydroxide or quaternary ammonium hydroxide), the associated acid of which has a pKa at least equal to 12 and advantageously to 13, to epoxides:

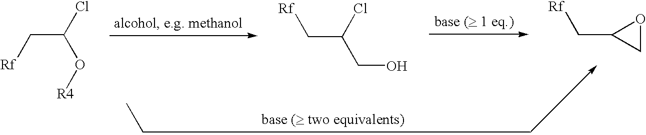

See the examples below.

The epoxides Rf-CHR₁CR′(O)CH₂ are important organic intermediates given the importance of“perfluoroalkylpropylene oxides and related compounds” in application on account of their common properties:

polymerization catalysts,

urethane-fluoropolyether acrylate copolymers,

treatment of metal surfaces,

transparent materials,

hydrophobic coatings,

cosmetics.

As has been stated previously, for reasons of ease of synthesis according to the invention and for reasons of stability, it is desirable for R_{f} not to bear in the omega position (i.e. at the other end of the chain) of the longest chain, a halogen heavier than fluorine (i.e. chlorine, bromine or iodine).

The nonlimiting examples that follow illustrate the invention.

### EXAMPLE 1 (COMPARATIVE)

Action of trifluoromethanesulfonyl chloride on enol ethers and esters in the presence of ruthenium activated with triphenylphosphine (for the operating conditions, see Kamigata et al., J. Chem. Soc. Perkin Trans., 1991, page 631, left-hand column).

| Substrate | θ° C. | T(h) | DC %^{(b)(1)} | DC %^{(a)} | RY %^{(b)} | Comments |
|---|---|---|---|---|---|---|
| 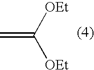 | 100° C. | 24.5 | 100 | nd^{(c)} | 0 | Formation with RY = 91.5% CF₃SO₂Et (characterization by ¹⁹F NMR and GC/MS). Detection by GC/MS of byproducts derived from (4): —CICH₂CH₂CO₂Et —CH₃CO₂Et |
| 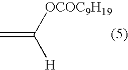 | 100° C. | 17.5 | 40 | 68 | 0 | Detection by GC/MS analysis of many decomposition products of CH₂═CHOCOC₉H₁₉: E.g.: C₉H₁₉CO₂H |
| | 120° C. | 16 | 38 | 87.5 | 0 | |
| 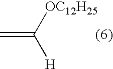 | 100° C. | 16 | 49 | 100 | 0 | Detection by GC/MS analysis of many decomposition products of CH₂═CHOC₁₂H₂₅: —C₁₂H₂₅OH (majority) —C₁₁H₂₃CHO —C₁₁H₂₃COCF₃ |

^{(a)}GC assay with internal standard

^{(b)19}F NMR assay with internal standard

^{(C)}not determined

⁽¹⁾relative to the sulfonyl chloride

### EXAMPLE 2

Action on various vinyl ethers

The initiator (0.376 mmol), the alkene (9.4 mmol) then triflyl chloride (9.4 mmol) are successively introduced into a 60 mm Schott tube at 20° C. The reaction is that indicated in the general equation of the description with R₁, R₂, R₃═H.

| Test (a) | Substrate | R | Initiator | t(h) | Substrate | DC %^{(b)} | RY %^{(b)} |
|---|---|---|---|---|---|---|---|
| RON 240B | 5 | OCOC₉H₁₉ | (PhCO₂)₂ | 13.5 | 1/1 | 82 | 41 |
| BJ 540A | | OCOC₉H₁₉ | (PhCO₂)₂ | 7 | 1/1 | 77 | 45 |
| BJ 544B | | OCOC₉H₁₉ | AIBN | 17 | 1/1.2 | 76.5 | 50 |
| BJ 545A | | OCOC₉H₁₉ | AIBN | 7 | 1/1.2 | 75.5 | 50 |
| BOA 137 | (12) | OCOCH₃ | AIBN | 7 | 1/1 | 82 | 52.5 |
| BOA 9 | (13) | (CH₂)₇CH₃ | AIBN | 7 | 1/1.1 | 91 | 77.5 |

The addition products were identified by fluorine-19 NMR and by gas chromatography coupled to a mass spectrograph.

The reaction was performed at 90° C. and, at this temperature, the reaction is complete, or at the very least has ended, after 7 hours.

Contrary to the rutheniumtriphenylphosphine chloride system, free-radical initiators allow the addition of trifluoromethyl, on the one hand, and of chloride, on the other hand, to the double bond of enol esters.

### EXAMPLE 3

Study of the role of the amount of free-radical initiators

One equivalent of triflyl chloride, AIBN in variable proportions and 1.2 equivalents of vinyl laurate are successively introduced into a 60-ml Schott tube, the reaction mixture is then maintained at 90° C. for 7 hours while the solution is stirred on a heating block.

The crude reaction mixture is analyzed by gas chromatography and fluorine-19 NMR.

| Mol % initiator | DC %^{(b)} | DC %^{(c)} | RY %^{(b)} |
|---|---|---|---|
| 0 | 70 | 57 | 25 |
| 1 | 77.2 | — | 43 |
| 4 | 84 | 86.5 | 56 |
| 6 | 85 | 95 | 54 |

### EXAMPLE 4 (COMPARATIVE)

Influence of the nature of the triflyl halide, reaction with perfluorobutanesulfonic fluorides

Under conditions similar to the previous test, perfluorobutanesulfonic fluoride is tested in place of the triflyl chloride.

| Substrate | R | DC %^{(b)} | DC %^{(c)} | RY %^{(b)} |
|---|---|---|---|---|
| (12) | —OAc | 27 | 79.5 | 0 |

Although the alkene reacts, the desired product is not obtained.

### EXAMPLE 5 (COMPARATIVE)

In the case of styrene

The above operating conditions were repeated, using styrene as the vinyl substrate. The results are collated in the table below.

| Test | DC %^{(a)} | DC %^{(a)} | Comments |
|---|---|---|---|
| BOA 1 | 65 | 10 | By ¹⁹F NMR and GC/MS the formation of |
| | | | the “dimer” is detected (2 diastereoisomers) |

Oligomerization or polymerization products are mainly formed.

### EXAMPLE 6

Comparison between vinyl ethers and vinyl esters

The above operating conditions were carried out on various substrates using triflyl chloride. The substrates used have different values of Z.

| Test | Z | DC %^{(a)} | RY %^{(a)} |
|---|---|---|---|
| BJ 542B | OC₁₂H₂₅ | 68 | 23.5 |
| BOA 142 | SPh | 91 | 22.5 |
| BOA 137 | OAc | 82 | 52.5 |
| RON 274 | OCOC₁₁H₂₃ | 85 | 54 |

### EXAMPLE 7

Unfavorable role of the phenyl radical

The above conditions were used on various substrates, with the value of Z being varied. The results are collated in the table below.

The ketone corresponds to the desired product after elimination of an acyl chloride.

| | DC | RY | Formation |
|---|---|---|---|
| Substrate | %^{(a)} | %^{(a)} | of ketone |
| CH₂═CH—OAc | 82 | 52.5 | — |
| CH₂═C(CH₃)—OAc | 78.5 | 0 | 50 |
| CH₂═C(Ph)—OAc | 69 | 1.5 | 5 |
| CH₂═C(CH₃)—OSiMe₃ | 89 | 0 | 28 |
| CH₂═C(Ph)—OSiMe₃ | 57.5 | 0 | 9 |

### EXAMPLE 8

### Experimental Section

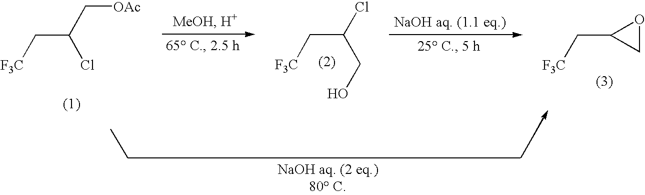

The acyl herein is such that Ac-O— is a propionate

### Synthesis Starting with 2-chloro-4,4,4-trifluorobutyl propionate (1)

15% sodium hydroxide (107 mmol) and 2-chloro-4,4,4-trifluorobutyl propionate (1) (10.03 g, 49.0 mmol) are loaded into a perfectly stirred and standardized 100-ml reactor equipped with a condenser and a thermometer. The reactor is placed in an oil bath at 90° C. with stirring at 320 rpm. The temperature of the medium reaches 84° C. After 24 minutes, the system is cooled with a bath of cardice. The medium is two-phase and the lower phase is colorless. GC allows the partial state of progress of the reaction to be monitored. The reactor is placed at 90° C. for 30 minutes at 520 rpm. The temperature of the medium reaches 84° C. The system is cooled in a bath of cardice. Monitoring by GC indicates the end of the reaction. The medium is two-phase and yellow. The lower phase is separated out by settling. 4.11 g (RY_{assayed}=46%) are recovered. In the same manner, a second batch is carried out (30 minutes with stirring at 520 rpm). 4.43 g (RY_{assayed}=43%) are recovered. The organic phases are distilled off under atmospheric pressure simply with a column head (t_{passage}=68-83° C.). 4.83 g of a colorless liquid are recovered (Pₘₐₛₛ=96%).

### Synthesis from the Alcohol (2): 2-Chloro-4,4,4-trifluorobutan-1-ol (2)

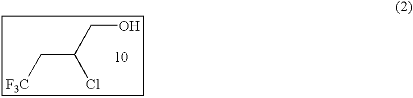

2-Chloro-4,4,4-trifluorobutyl propionate (1) (9.58 g, 95.8 mmol), methanol (92 ml, 2.3 mol) and concentrated sulfuric acid (0.53 g, 10 mmol) are loaded into a 250-ml three-necked flask equipped with a condenser, a thermometer, a septum and a magnetic stirrer. The reaction medium is heated to reflux (between 60 and 65° C.). The mixture is left stirring for 2.5 hours. Distillation is performed at atmospheric pressure to remove the methanol and the methyl acetate. The residue is distilled at reduced pressure (38 mmHg). The following are collected:

1st between 38 and 58° C.: 0.318 g at more than 99% pure

2nd between 60 and 66° C.: 10.15 g at more than 99% pure

3rd between 57 and 35° C.: 0.63 g at more than 95% pure.

11.1 g are thus finally collected. Assay by 19F NMR gives a RY_{isolated} (2)=69%.

b.p.=60-65° C./38 mmHg

### 4,4,4-Trifluoro-1,2-epoxybutane (3)

15% sodium hydroxide (37 mmol) and 2-chloro-4,4,4-trifluorobutan-1-ol (2) are loaded into a perfectly stirred and standardized 100-ml reactor equipped with a condenser and a thermometer. The mixture is stirred at 20° C. for 1 hour 40 minutes at 250 rpm. A two-phase medium is obtained. GC of the mixture indicates a virtually complete DC. The mixture is allowed to settle for 2 hours. The two phases are separated. The organic phase is washed with 5 ml of H₂O. 3.1 g of crude product are recovered. ¹⁹F NMR analysis gives an RY_{assayed} (3)=68%

b.p.=68-69° C./627 mmHg

### EXAMPLE 9

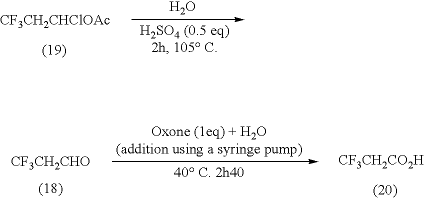

### 3,3,3-Trifluoropropanal (18)

1-Chloro-3,3,3-trifluoropropyl acetate (19) (10.02 g, 52.7 mmol), water (51 ml) and 95% sulfiric acid (1.5 ml, 26.6 mmol) are loaded into a 100-ml four-necked flask equipped with a magnetic stirrer, a cardice trap (connected to an antireturn bubbler) and a thermometer. The mixture is heated to 105° C. When the temperature of the medium reaches 87.5° C., a reflux appears. The temperature falls by a few degrees (83° C.). The mixture is heated for 2 hours. It is then cooled to ambient temperature and the trap is kept. The acetone in the trap is replaced with warm water. The trap is washed with water and the washing liquors are combined with the reaction medium. Assay by ¹⁹F NMR gives an RY_{assayed} (18) of 89%. The 3,3,3-trifluoropropanal (18) is stored in the acidic aqueous medium.

### 3,3,3-Trifluoropropionic acid (20)

1-Chloro-3,3,3-trifluoropropyl acetate (19) (1.0 g, 5.3 mmol), water (5 ml) and 95% sulfuric acid (0.15 ml, 2.6 mmol) are loaded into a 50-ml three-necked flask equipped with a magnetic stirrer, a cardice trap (connected to an antiretum bubbler), a thermometer and a Teflon tube. The mixture is heated to 105° C. When the temperature of the medium reaches 92° C., a reflux appears. The temperature falls by a few degrees (87° C.). The mixture is heated for 2 hours. It is then allowed to cool to ambient temperature, and the trap is kept. The acetone in the trap is replaced with warm water. The trap is washed with a minimum amount of water. Assay by ¹⁹F NMR gives an RY_{assayed} (m) of 73% (3.88 mmol). The solution of Oxone® (2.25 g, 3.67 mmol) in 10 ml of water is prepared. This solution is added by syringe pump over 30 minutes at 40° C. The mixture is left at 40° C. for 2 hours 15 minutes. 2.07 mmol of acid are obtained with an RY_{oxidation} (20)=73%.

### EXAMPLE 10

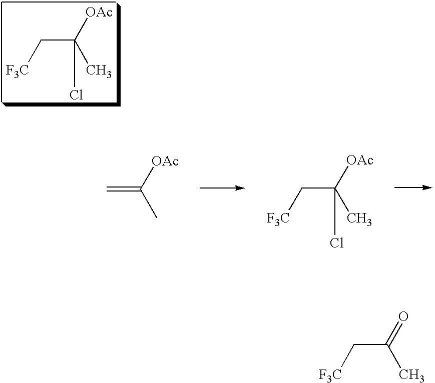

relative to:

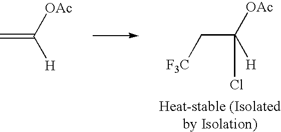

The presence of a CH₃ unit is not an inconvenience for the trifluoromethylation reaction. However, the product of vicinal-chlorotrifluoromethylation is not isolated; as a result of the fact that there is now a tertiary carbon instead of a secondary carbon—as in CF₃CH₂CHClOAc—this reaction brings about a predominant elimination of acetyl chloride:

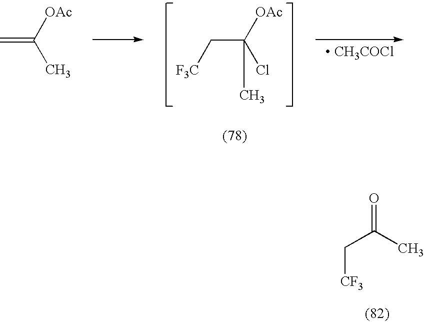

As shown by the results below, the intermediate (78) was detected by working at lower temperature and by analyzing the reaction medium after reaction for 1 to 3 hours:

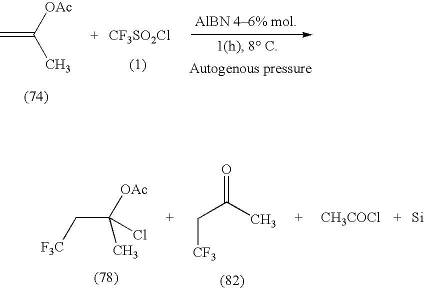

| Test | (1)/(74) | θ° C. | t(h) | DC %(1)%^{(b)} | DC %(74)%^{(a)} | RY(78)%^{(b)} | RY(82)%^{(b)} |
|---|---|---|---|---|---|---|---|
| BJ665 | 1/1 | 80 | 1 | 60 | 65 | 28.5 | 7.5 |
| | | | 2 | 68 | 75.5 | 21 | 19 |
| BJ666 | 1/1 | 90 | 1 | 69 | 78 | 20 | 23 |
| | | | 2 | 72 | 90 | 5 | 39 |
| | | | 3 | 76 | >95 | 1 | 39.5 |

^{(a)}GC assay with internal standard,
^{(b)}assay by ¹⁹F NMR with internal standard: (78): δ = 7.84 ppm (/TFA) and (82): δ = 8.97 ppm (/TFA)

Another object of the invention is to provide a precursor family of these nitrogen-containing heterocyclic compounds. Use of these precursors should be easy, and should not require numerous stages.

Another object of this invention is to provide a process of synthesis of said precursors.

Another object of this invention is to provide a technique for utilization of these precursors.

These objects, and others will appear below, are achieved via compounds of formula (I):

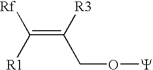

in which:

R_{f} is a radical that carries a perfluoromethylene group, and said group ensures bonding to the remainder of the molecule;

R₁ and R₃, which can be the same or different, are chosen from among hydrogen and alkyl or aryl radicals;

Ψ is an electroattractor group such that Ψ-O—H is an acid whose pKa (in water) is at most equal to 8, advantageously 6, and preferably 5.

According to this invention, it is desirable that the —O—H group comprises an acid whose pKa is at least equal to 1, advantageously 2. Actually, it is desirable that the —O′ group does not constitute a good starting group, as much for use as a precursor of cycloaddition as for its synthesis.

It is desirable that RI and R₂ do not unduly overload the molecule; also it is a good idea to prevent R₁ and/or R₃ being attached to the double bond by a tertiary carbon, or even a secondary carbon.

Thus, it is preferable that at least one of R₁ and R₃ be a light alkyl (light, i.e., with at most 4 carbons), or even better, a hydrogen.

Thus, it is preferred that R₁ be hydrogen, it is likewise preferred that R₃ be hydrogen; and it is even more preferred that R₁ and R₃ be hydrogens.

Advantageously, R_{f} has formula (II)

GEA-(CX₂)ₚ—

where:

the X, similar (i.e., they are identical) or different, represent a chlorine, a fluorine, or a radical of formula CₙF₂ₙ₊₁ with n an integer that is at most equal to 5, preferably 2, with the condition that the Xs of the methylene group that carry the open bond are not chlorine and that at least one of them is either a fluorine atom, advantageously the Xs of the methylene that carry the open bond, or else are fluorine atoms or else a fluorine atom and a radical of formula CₙF₂ₙ₊₁ (such radicals are actually considered to be electronically related to fluorine atoms);

GEA is a hydrocarbon group or electroattractor group (i.e., the Hammett constant σₚ (sigma p) is >0, advantageously at least equal to 0.2), preferably inert, advantageously when p is equal to 1, an electroattractor group (cf. lines above);

p is a positive integer, i.e., it cannot have a value of 0.

GEA is advantageously fluorine, especially when p is less than or equal to 2.

The Xs are advantageously all fluorines, especially when p is less than or equal to 2.

Another value of GEA (electroattractor group) is the chlorine value; in this case, GEA is a chlorine.

p represents an integer that is advantageously at most equal to 4, preferably 2.

GEA advantageously represents an electroattractor group whose possible functions are inert under reaction conditions, advantageously fluorine or a perfluorinated radical of formula CₙF₂ₙ₊₁ with n an integer that is at most equal to 8, advantageously 5.

The total number of carbons of R_{f} is advantageously between 1 and 15, preferably between 1 and 10.

It is advantageous that R_{f} has formula CᵣF₂ₙ₊₁, with r a positive integer from 1 to 10, advantageously from 1 to 5, preferably from 1 to 3.

This invention is particularly advantageous for the R_{f} of low molecular weight, i.e., those that are relatively volatile (i.e., corresponding to an R_{f}H whose boiling point under atmospheric pressure is at most equal to 100° C.). The technique is particularly advantageous for the R_{f} that have a radical with an odd number of carbons, and particular mention should be made of R_{f} in C₁, C₂ and C₃. Thus, the trifluoromethyl, pentafluoroethyl and heptafluoropropyl radicals are included among the preferred values of R_{f}.

According to this invention, it is preferable that represents an acyl, advantageously such that the pKa (measured or reduced to the value in water) is a least equal to roughly 2 (the expression roughly is used here to highlight the fact that the number that follows it corresponds to a mathematical rounding), advantageously to roughly 3.

Usually, —O—-H is an alkanoic acid, advantageously with 1 to 8 carbon atoms, preferably from 2 to 5.

Economically, the value of that is equal to acetyl is the most advantageous.

Just as was mentioned above, another object of this invention is to provide a means of easy access to the above-mentioned molecules.

This object is achieved via a process of synthesis of the compound of formula (I), which entails bringing into contact a compound of formula (III):

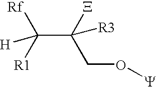

(where [symbol] (xi) represents a starting halogen group (heavier than fluorine, advantageously chlorine or bromine) or a pseudohalogen group, such that [symbol] H exhibits a Hammett constant that is at least equal to that of trifluoroacetic acid, advantageously at least equal to that of mesylic acid) with a base that is chosen from among strong nitrogen-containing bases, of which the associated acid has a pKa that is at least equal to 12 among the anionic bases with the condition that when the base is a non-nitrogen-containing anionic base, the latter is in the presence of a solvent or a mixture of polar solvents.

To date, access to compounds of formula (1) has not been described; a good reason for this is that the most direct approach based on known compounds is difficult and generally leads to products other than the one desired (see in particular the comparative examples).

The compounds of formula (III) are delicate compounds, purification of which is often difficult, indeed almost impossible, due to instability, especially thermal, of the compounds of formula (III), mainly when [symbol] represents a halogen that forms a good starting group such as bromine or iodine.

The chlorinated derivative is slightly more stable.

The technique of synthesizing compounds of formula (III) was described in international application PCT WO 01/58833, filed in the name of the applicant.

Generally, the compounds of formula (III) can be synthesized by the reaction that is defined by the following equation:

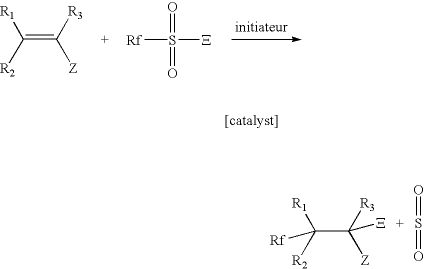

with Z representing a methylene group (optionally substituted, but preferably not substituted) that carries Ψ-O— and [symbol] limited here to Cl and Br, and even I (but sulfonyl iodide is not stable; its existence, which could only be temporary, has not been demonstrated and cannot be implemented, except for being made in situ).

The extraction of acid H [symbol] from the molecule of formula (III) to yield the molecule of formula (I) is difficult to control, most of the bases yielding nucleophilic substitution or degradation reactions, and even elimination of a halogen from group Rf.

Following the research that led to this invention, it has been shown that when choosing certain bases, the reaction could have taken place in the desired direction. It has likewise been shown that other bases could be used under the condition of choosing the operating conditions, specifically the reaction medium.

It can thus be stated that the following bases can yield good results:

nonanionic bases in which a nitrogen doublet is conjugated with a double carbon-nitrogen bond; these bases can be used either alone in stoichiometric or superstoichiometric amounts, or can be used in catalytic amounts with another base, preferably more basic than said nonanionic bases; in particular these bases can be used with the following bases, which are used in principle in stoichiometric or superstoichiometric amounts;

anionic nitrogen-containing bases that yield good results, but are generally costly, and whose effect is all the better provided aprotic polar solvents are used as solvents;

non-nitrogen-containing anionic bases, advantageously not derived from alcohol or water (the hydroxide and alcoholate ions are respectively prohibited and preferably should be avoided). These bases do not yield good results when a polar solvent is used whose donor index is at least equal to 10, advantageously 15, and preferably 20.

When the polar solvents are used according to the process of the invention, it is preferable that these polar solvents have a dielectric constant \acute{ε}(epsilon) that is at least equal to 7. Moreover, when the solvent is basic, i.e., when it has an elevated donor number (greater than 20), it is preferable that this basicity be relatively weak in terms of the Bronsted basicity, i.e., that the pKa of the acid associated with said solvent be greater than 5, advantageously 6, and more preferably 7.

Advantageously, said reaction medium is aprotic and anhydrous. In particular, it is desirable that this aprotic and anhydrous medium be such that the strongest acid that is present in the medium, ignoring the substrate, has a pKa of at least 20, better 25, advantageously 30, and preferably 35. This limitation is intended to prevent parasitic reactions at the time of removal of the proton from the substrate by the base; in fact, the anions that are obtained from the removal of a proton by a base are nucleophiles, which can lead to a nucleophilic substitution reaction that is undesirable.

Thus, it is preferable that such anions are not formed. More generally, it is preferable that the components of the reaction mixture are not, upon contact with the base used, able to provide nucleophilic anions. Strong acids (pKa≦2), and even medium acids (2<pKa≦4.5) do not lead, strictly speaking, to a reaction since they use up the base by yielding only a few nucleophilic anions or none at all, and therefore few parasitic reactions or none at all. The acids associated with the bases according to this invention, or derived therefrom, apparently do not prejudice this invention. This is why hydroxide ions, and even alcoholate ions, are not suited to the processes according to this invention. Actually, they lead to molecules that are to be avoided as solvents.

Thus, it is preferable that, in the reagent, the content of unstable hydrogen atoms (i.e., those that do not correspond to the pKa indicated above) is at most equal to ⅓, advantageously ¼, preferably 10% (in moles), relative to the initial content of that of said base or said compound of formula (III) that is not in excess.

One of the advantages of the cryptants is to make it possible to remove at least partially the solvents with a strong donor index.

For a definition of the donor index (or donor number), refer to the work of Christian Reichardt, Solvents and Solvent Effects in Organic Chemistry, p. 19 (1988), a work where the negative of the enthalpy (—H expressed in kilocalories/mole) of the interaction between the solvent and the antimony pentachloride in a diluted solution of dichloromethane is found as a definition.

In the case of mixtures of solvents or of solvents containing cryptants, the donor index will be calculated by a donor function by multiplying the donor index of each of the solvents by the molar fraction that it represents and by adding these products.

Among the anionic nitrogen-containing bases, the salts, especially alkaline or alkaline-earth salts, amines that may or may not be silylated, as well as silyl amines, should be cited. Among the salts yielding the best results, salified disilylamines and especially salts, in particular alkaline or alkaline-earth salts, of hexamethyldisilazane (IMDZ) should be cited.

Among the non-nitrogen-containing anionic bases, the non-oxygenated bases, such as, for example, alkaline hydrides or alkaline-earth hydrides, and alkane salts, such as butyl lithium and alkaline carbonates, should be cited. It should be remembered that the bases of this family should be used in the presence of polar solvents.

Let us remember that it is desirable to use solvents that can be easily distilled (Eb less than or equal to 120° C.) and/or that are water-miscible, advantageously in any proportion in order to more easily treat the reaction mixture.

The bases yielding the best results are the bases comprising 2 conjugated nitrogen atoms, as has already been mentioned and as is presented in detail below.

According to one advantageous embodiment according to this invention, said base contains nitrogen and is nonanionic and corresponds to formula (IV):

D-A′=A-R2

where A is a metalloid atom of column VB (the column of nitrogen, and advantageously the latter) (the periodic table that is used in this application is the one from the supplement to the Bulletin of the Chemical Society of France, January 1966, No. 1).

where A″ is a carbon atom that carries hydrogen or is substituted by a hydrocarbon radical R₅,

where the radical D is:

either a doublet-carrier metalloid chosen from among:

the chalcogens that are advantageously monosubstituted by a monovalent radical R₆ (in which case the chalcogens constitute said doublet-carrier metalloid),

the metalloids of column VB, especially nitrogen or phosphorus (in which case the metalloids of column V constitute said doublet-carrier metalloid), preferably nitrogen, which metalloids of column VB are advantageously disubstituted by two monovalent hydrocarbon radicals R₆ and R′₆ to form a radical D of formula -A′(R₆)(R′₆);

or else a carrier radical, at the same time a metalloid atom and at least one unsaturation, said unsaturation or unsaturations ensuring conjugation between a doublet of said metalloid atom and the double bond -A3=A-; this group is linked to the remainder of the molecule by a single bond carried by an atom that is chosen from among the hybridization carbon atoms sp² substituted by a function or a divalent radical R₇ that carry a hydrogen or are possibly substituted by a carbon radical R₆.

It should be remembered that in this formula, the metalloids of column VB are preferably a nitrogen, either for A″ or for A′.

When A″ is an atom of column VB, and specifically a nitrogen, it is preferred that D be chosen from among those described above and of which the single bond that ensures the link to the remainder of the molecule is carried by an atom that is chosen from among the hybridization carbon atoms sp² substituted by a function or a bivalent radical R₇ that carries a hydrogen or is possibly substituted by a carbon radical R& to yield a formula of D that is specified below:

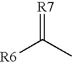

When said carbon carries a hydrogen, this hydrogen is found in place of R₆ thus to impart the hydrogen value to R₆.

As was stated above, it is desirable that the base of formula (IV) comprise a metalloid atom (saturated, i.e., does not carry the double bond), exhibiting resonance (or conjugation) with a n bond linking two atoms, of which at least one is a disubstituted and positively charged atom from column VB; advantageously, an organic base comprising a trivalent atom of column VB (column of nitrogen in the periodic table), advantageously of nitrogen, an atom of which the doublet is conjugated directly or indirectly with a π bond linking two atoms, of which at least one is an atom of column VB (namely A).

According to a particularly advantageous embodiment of this invention, said π bond linking two atoms is the π bond of an imine function (>C═N—).

This imine function can be written as follows:

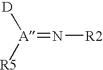

with A″ representing a carbon,

with D chosen from among:

the chalcogens that are monosubstituted by a monovalent R₆ radical,

a metalloid of column VB, nitrogen advantageously substituted by two monovalent radicals R₆ and R′₆ specifically, or phosphorus, preferably nitrogen; and

* those described above where the link to the remainder of the molecule is ensured by a bond carried by the atoms of carbon sp² substituted by a function or a divalent radical R₇ that carries a hydrogen or is possibly substituted by a carbon radical R₆.

The R₅ radical is chosen from among hydrogen, the values of D, and among the hydrocarbon radicals, advantageously aryls and mainly alkyls.

It is preferable that the D radical and this imine function be arranged such that the atoms of nitrogen and said metalloid be as distant as possible, in other words and for example that the nitrogen of the imine function is that of the two atoms linked by the i bond that is the most distant from the trivalent atom of column V. The aforementioned regarding the imine function is generally applicable to all atoms of the VB column linked by the π bond in the case where the π bond comprises an atom of carbon and an atom of column V.

According to this invention, it is preferred that the organic cation comprising a trivalent atom of column VB whose doublet is conjugated with a π bond has a scheme, or rather a skeleton, of formula >N—[C═C]ᵥ—C═Ni^{±}<, with v equal to zero or an integer chosen in a closed interval (i.e., inclusive) 1 to 4, advantageously from 1 to 3, and preferably from 1 to 2. Preferably the preceding sequence corresponds to formula:

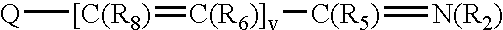

with Q representing

a chalcogen substituted by an aliphatic or aromatic radical R₉; or

a disubstituted phosphorus or more preferably a nitrogen disubstituted by two radicals, the same or different, aliphatic or aromatic R₉ and R₁₀: (R₁₀)(R₉)N—;

with v equal to zero or an integer that is chosen in a closed interval (i.e., inclusive) 1 to 4, advantageously from 1 to 3, preferably from 1 to 2 and where R₂ is chosen from among the hydrocarbon derivatives, advantageously alkyls of at most 4 atoms of carbon, and hydrogen.

Advantageously, according to this invention, said trivalent atom of column VB forms or constitutes a tertiary amine.

More specifically, it is desirable that said organic base comprising a trivalent atom from column VB, whose doublet is conjugated with a 7r bond, constitutes a molecule of the following formula:

(R₁₀)(R₉)N—[C(R₈)═C(R₆)]ᵥ—C(R₅)═N—(R₂)

with v equal to zero or an integer chosen in a closed interval (i.e., inclusive) 1 to 4, advantageously from 1 to 3, preferably from 1 to 2, and where R₂, R₅, R₆, and R₈, the same or different, are chosen from among the hydrocarbon groups, advantageously alkyls of at most 4 atoms of carbon, and hydrogen, and where R₁₀ and R₉, the same or different, are chosen from among the hydrocarbon groups, advantageously alkyls of at most 4 atoms of carbon, one or two of the substituents R₂, R₅, R₆, R₈, R₉ and R₁₀ possibly being linked to (an)other remaining substituent(s) to form one, two or several cycles, especially aromatic, see below.

The potentialization effect of the base is particularly conspicuous when said bond linking two atoms is intracyclic (or a mesomeric form is intracyclic), even when it is intracyclic in an aromatic cycle.

This is especially the case of pyridine and diazine (preferably metadiazine, see formulas below) cycles and of cycles that are derived therefrom like quinoline or isoquinoline, such as, for example:

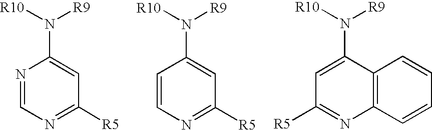

More specifically, the organic base comprising a saturated metalloid atom that has resonance with a bond can be advantageously chosen from among the dialkylaminopyridines, especially in the para- or ortho-position (i.e., in 2-position of pyridine or 4-position, see formula above).

Thus, said base that carries at least 2 trivalent nitrogens is advantageously such that said 2 trivalent nitrogens form a bond system comprising an imine that is conjugated with the doublet of an amine.

The amines, such as DBU (DiazaBicycloUndecene, which has 9 carbon atoms) or DBN (DiazaBicycloNonene, which has 7 carbon atoms), that form with the imine function a substituted amidine function, advantageously intracyclic with 1 or even 2 cycles, also constitute bases that are particularly advantageous for implementing this invention. Examples of such bases can be found in the diazabicycloalkenes with 6 to 15 carbon atoms:

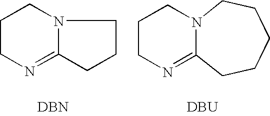

The cycles with 5 links are also advantageous when they have two or three heteroatoms.

For example, structures of the cyclic imidazole, oxazole, or guanidine type, or even indolic type:

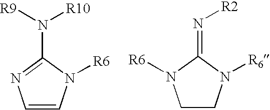

R₆′ and R₆″ have the same value as R₆.

It is possible to substitute the free aryl (involved in an aromatic compound) or aliphatic (of which the connection point is a carbon sp³) vertices. This is of relatively little advantage, however, and has the drawback of loading the base.

Triazole structures can also be envisaged:

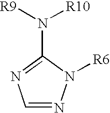

The pyrazole structures are also possible.

It should also be mentioned that among the noncyclic structures, there may be a certain advantage in using guanidine structures that have the characteristic of being easily derived from guanidine and of having a highly resonant formula:

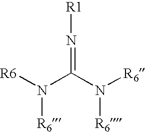

where R₆′″ and R₆″″ are chosen from among the same values as R₆; they can be the same or different from other R₆, as well as R₂. It is preferable, if compounds with a low melting point are desired, that the molecule be dissymmetrical. R₆′″ and R₆″″ can be interlinked to form cycles, advantageously aromatic. It is desirable that the molecular mass of the base be at most equal to 300, advantageously 250, preferably 200. When the bases are polyfunctional (i.e., carry several basic systems such as are described above), these values should be reduced by the basic function D-A″=A-R₂.

Another object of this invention is to find a technique for cycloaddition that can work with compounds of formula (1).

During the research that led to this invention, it was possible to demonstrate that, under the condition of exposure to relatively mild conditions, i.e., to a temperature of less than 150° C., preferably at most equal to 100° C., the cycloadditions, or cyclocondensations, with a co-substrate that carries 2 double bonds yield good results and that their use yields cyclic compounds, notably heterocyclic compounds that are substituted by an R_{f} group.

Although it is possible to carry out cycloadditions of type 2+4, one of the main advantages of this invention is to provide easy access to heterocyclic compounds by addition of type 3+2, and especially of dipolar cycloadditions 1,3. Advantageously, the co-substrate is an organic compound that carries a pentavalent nitrogen that itself carries 2 double bonds (including donor-acceptor type bonds), of which at least one double bond links said nitrogen to a carbon. In particular, the co-substrates that provide the portion of 3 atoms correspond to the following canonic dipolar forms:

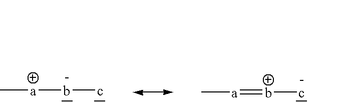

Here, b can be a nitrogen, possibly substituted, a and c can be oxygen, nitrogen, or carbon, and the latter two atoms can carry a hydrocarbon radical or a hydrogen.

For this type of reaction, reference is made to the general work Advanced Organic Chemistry, Third Edition, by Jerry March, page 743 ff, and to the documents that are cited in this reference book.

Heating of these compounds, whether in the presence of a solvent or not, yields a cycloaddition even without a catalyst. Nevertheless, some of the compounds whose canonic form was mentioned above should be synthesized in situ.

The nonlimiting examples below illustrate the invention.

### EXAMPLE 1

### Dehydrohalogenation of the Precursor CF₃CH₂CHClCH₂OAc

Here, R_{f} is trifluoromethyl, R₁ and R₃ are hydrogen, and is chlorine.

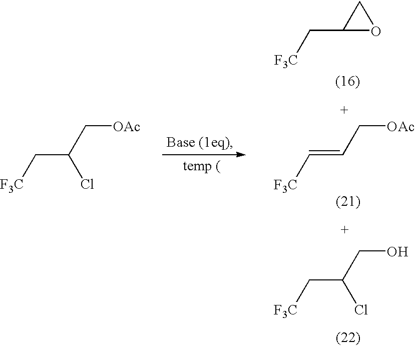

The equation of the reaction is given below with some of the impurities identified:

| Bases (pKa | | | | | | | | | | |
|---|---|---|---|---|---|---|---|---|---|---|
| of the | (3a) | Base | | | | TT^{(a)} | | | | |
| associated acid) | mmol | eq | Solvents | t (h) | Temp. (° C.) | (3a)% | RR^{(a)} (16)% | RR^{(a)} (21)% | RR^{(a)} (22)% | RT % |
| DBU(12) | 1.1 | 1 | Diisopropyl Ether | 17 | 0^{(b)} at ambient | 59 | 0 | 49.1 | 0 | 83.22 |
| | | | ND˜18 | | temp. | | | | | |
| DBU(12) | 1.1 | 1 | Diisopropyl Ether | 4 | 50 | 69.2 | 0 | 71.2 | 0 | 102.89 |
| DBU(12) | 1.1 | 1 | Diisopropyl Ether | 4 | 70 | 91 | 0 | 77.5 | 0 | 85.16 |
| Et₃N(10.8) | 1.1 | 1 | Diisopropyl Ether | 17 | 20 | 14.5 | 0 | 0 | 0 | 0.00 |
| Et₃N | 1.1 | 1 | Diisopropyl Ether | 4 | 50 | 5.8 | 0 | 0 | 0 | 0.00 |
| dabco | 1.1 | 1 | Diisopropyl Ether | 4 | 50 | 4.75 | 0 | traces | 0 | 12.63 |
| NaH(31) | 2.44 | 0.9 | PhCl | 4 | 0^{(c)} at ambient | 41.1 | 2.2 | 0 | 25.2 | 0.00 |
| | | | | | temp. | | | | | |
| NaH(31) | 0.98 | 0.9 | THF (ND = 20) | 4 | 0^{(c)} at ambient | 67 | 1.5 | 59.5 | 7.85 | 88.81 |
| | | | | | temp. | | | | | |
| KHMDZ | 1.8 | 0.6 | THF | 4 | 0^{(c)} at ambient | 47.5 | 0 | 42 | 0 | 88.42 |
| (27) | | | | | temp. | | | | | |
| MeONa(15) | 2.24 | 1 | Diisopropyl Ether | 4 | 0^{(b)} at ambient | 96 | 3.7 | traces | 23.3 | 2.60 |
| | | | | | temp. | | | | | |
| MeONa | 2.80 | 0.8 | Diisopropyl Ether | 4 | 0^{(b)} at ambient | 84.8 | n.d.^{(d)} | n.d.^{(d)} | 56.8 | nd |
| | | | | | temp. | | | | | |
| MeONa | 1.54 | 1.4 | MeOH | 4 | 0^{(c)} at ambient | 100 | 10.2 | 0 | 60.6 | 0.00 |
| | | | | | temp. | | | | | |

^{(a)}NMR ¹⁹F with an internal standard
^{(b)}addition at ° C. then it is allowed to react at ambient temperature
^{(c)}addition below 0° C. and is left in an ice bath for ½ hour and then allowed to react at ambient temperature
^{(d)}(16) and (21) could not be separated by NMR ¹⁹F (16) + (21) = 12.3%.

### EXAMPLE 2

### Dehydrohalogenation of the Precursor CF₃CH₂CHClCH₂OAc in the presence of DMF

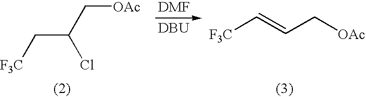

| Batches | RR(Isolated) % |
|---|---|
| (2): 1830 g [8.95 mol] | 80% |
| DBU: 1500 g [9.85 mol] | |
| DMF: 7.5 liters | |
| 25° C., 2 to 3 hours | |

### EXAMPLE 3

### Dehydrohalogenation of the Precursor CF₃CH₂CHClCH₂OAc by means of Potassium Carbonate

It has been demonstrated that potassium carbonate makes it possible to carry out this reaction of dehydrochlorination no longer at 25° C., but at 60° C. and in dipolar aprotic solvents such as DMF or NMP (ND=27.3):

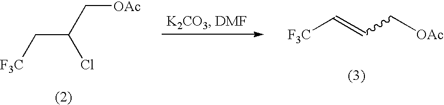

### Effect of Concentration

(DMF, K₂CO₃ (5 eq.) 60° C., 4 hours)

| % by weight^{(a)} | TT(2) %^{(b)} | RR(3) %^{(b)} |
|---|---|---|
| 11% | 100 | 92% (E + Z) |
| 16.5% | 100 | 85.5% (E + Z) |

^{(a)}Mass of (2)/mass of DMF
^{(b)}Metering by NMR ¹⁹F with an internal standard.

### Effect of K₂CO₃

| K₂CO₃ (nbe eq) | TT (2)^{(a)} (% mol) | RR (3)^{(a)} (% mol) |
|---|---|---|
| 5 | 100 | 88 + 10 |
| 3 | 100 | 86 + 9 |
| 1.5 | 99 | 87 + 9 |

^{(a)}Metering by NMR ¹⁹F with PhOCF₃ as an internal standard.

Operating conditions: 60° C., 'h, 3 g (15 mmol) of (2) for 28.5 g of DMF or as a function of the stoichiometry in K₂CO₃ between 7 and 8.7% by weight.

### EXAMPLE 4

### Dehydrohalogenation of the Precursor CF₃CH₂CHBrCH₂OAc

The dehydrobromination reaction is carried out without difficulties according to the same principle:

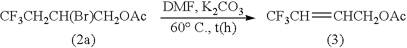

| Temps^{(a)} | (2a)^{(b)} | (3E)^{(b)} | (3Z)^{(b)} |
|---|---|---|---|
| 45 min | 52.2 | 43 | 4.8 |
| 2 h | 31.4 | 61.7 | 6.9 |
| 3 h | 18.7 | 72.9 | 8.2 |
| 4 h | 12.5 | 78.5 | 8.9 |
| 5 h | 7.8 | 82.9 | 9.3 |
| 6.5 h | 2.4 | 87.6 | 9.9 |

^{(a)}Test 02JGR910: test in RPAS 100 ml with double jacket, stirring: 4 inclined blades (600 rpm): 2a (10 g at 96% by weight, 38.5 mmol), DMF (35 g), K₂CO₃ (18 g, 0.130 mol),
^{(b)}% CPG area

The reaction medium is left for one night at ambient temperature before treatment.

After filtration (filter glass no. 4) and washing of the cake with 10 ml of DMF, the filtrate is run on 20 ml water and this aqueous phase is extracted with MTBE (3*25 ml). The organic layer is metered by NMR ¹⁹F with an internal standard:

| CF₃CH₂— | CF₃CH═CHCH₂Oac | CF₃CH═CHCH₂OAc |
|---|---|---|
| CHBrCH₂OAc | (E) | (Z) |
| TT = 100% | RR = 74.5% | RR = 8% |

### EXAMPLE 5

### Cycloaddition Involving CF₃CH═CHCH₂OAc

Synthesis of original heterocyclic compounds

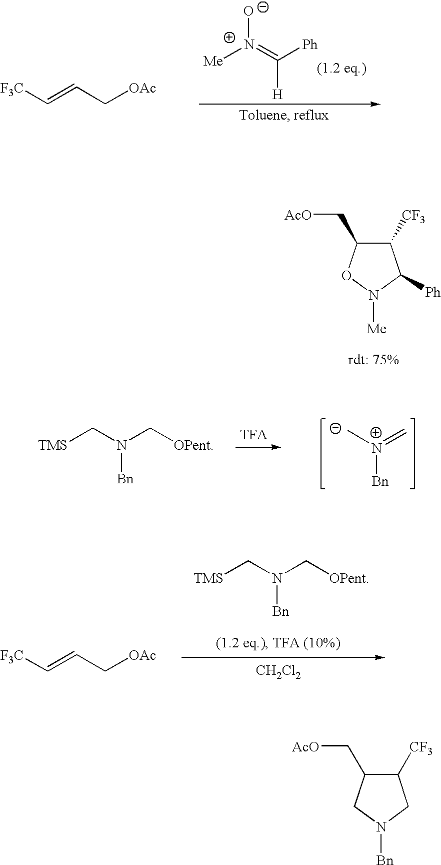

### EXAMPLE 6

### Dehydrochlorination with DBU

Synthesis of CF₃CH═CHCH₂OAc

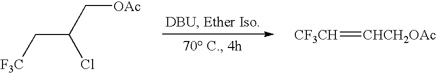

A 250 ml three-necked flask that is equipped with a coolant, a thermometer, an addition ampule and magnetic stirring is filled with 2-chloro-4,4,4-trifluorobutyl acetate (3a) (12 g, 58.9 mmol) and diisopropyl ether (135 ml, 0.95 mol). 1,8-diazabicyclo[5.4.0]undec-7-ene (8.98 g, 59 mmol) is added drop by drop within 25 minutes. The reaction medium is heated to 70° C. for 4 hours. This development is followed by injections of CPG. At the end of the reaction, a formed product yield of 77.5% is determined by CPG metering with an internal standard.

The reaction medium is poured into 20 ml of water. Three times 100 ml of diisopropyl ether is extracted, and the organic phases regrouped beforehand are dried on magnesium sulfate. After concentration of diisopropyl ether at atmospheric pressure, 7 g (yield: 71 %) of the trifluorobutenol acetate is isolated by standard distillation.

### Example of Dehydrobromination with K₂CO₃

A double-walled 100 ml reactor with mechanical stirring is filled with anhydrous NMP (10.25 g), potassium carbonate (5.13 g, 37.15 mmol), and then CF₃CH₂CHBrCH₂OAc (9.13 g (78% by weight), 28.6 mmol (1 eq.)). The reaction medium is brought to 60° C. for 4-6 hours. The development of the reaction is followed by CPG (TT=100%, RR=95%).

At the end of the reaction, the trifluorobutenol acetate is distilled directly from the reaction medium. Peb.=45° C. at 30 mbar.

### Structural Characterization

### Characteristic Absorbances of the IR Spectrum

1750 cm⁻¹ vC═O and 1230 cm⁻¹ vC═O acetate function

1690 cm⁻¹ vC═C and C—H (out of plane) unsaturation of the trans type

1127 cm⁻¹ vC—F

### Chemical Displacements of Resonance Lines of the Proton, of fluorine-19 and of carbon-13 (in CDCl₃)

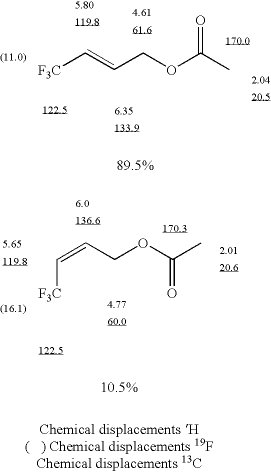

### EXAMPLE 7

### Development of CF₃CH═CHCH₂OAc

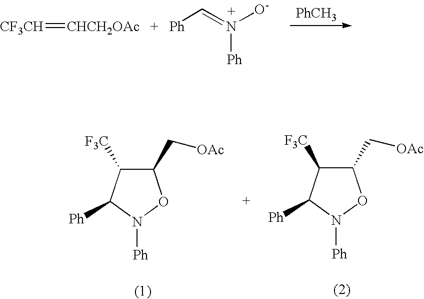

A 10 ml vessel provided with coolant is filled successively with the following at ambient temperature: 213 mg (1.27 mmol) of 4,4,4-trifluorobutenol acetate (93/7 E/Z, 100% by weight), 3 g of toluene and 228 mg (1.12 mmol) of N,-diphenylnitrone (97% by weight). The solution is brought to solvent reflux (the nitrone is then rapidly solubilized) for 26 hours. The toluene and excess trifluorobutenol acetate are evaporated under reduced pressure, and a mixed product of stereoisomers is obtained with a yield of 87% (ratio: (1)/(2), 25/1)

What is claimed is:

1. A compound of formula (I) 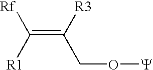
in which
R_{f} is a radical that carries a perfluoromethylene group, which group ensures bonding to the remainder of the molecule;
R1 and R3, which can be the same or different, are chosen from among hydrogen and alkyl or aryl radicals; and
Ψ is an electroattractor group such that Ψ-O—H is an acid whose pKa (in water) is at most equal to 8.

2. A compound according to claim 1 wherein Ψ is an electroattractor group such that Ψ-O—H is an acid whose pKa (in water) is at most equal to 6.

3. A compound according to claim 1 wherein Ψ is an electroattractor group such that Ψ-O—H is an acid whose pKa (in water) is at most equal to 5.

4. A compound according to claim 1, characterized by the fact that Ψ is an electroattractor group such that Ψ-O—H is an acid whose pKa (in water) is at least equal to 1.

5. A compound according to claim 1, characterized by the fact that Ψ is an electroattractor group such that Ψ-O—H is an acid whose pKa (in water) is at least equal to 2.

6. A compound according to claim 1, wherein at least one of R1 and R3 is a light alkyl, or a hydrogen.

7. A compound according to claim 1, wherein at least one of R1 and R3 is a hydrogen.

8. A compound according to claim 1, wherein the R_{f} radical has the following formula:
GEA-(CX₂)ₚ—
where
X, the same or different, represent a chlorine, a fluorine, or a radical of formula CₙF₂ₙ₊₁ with n an integer that is at most equal to 5, preferably 2, with the condition that the X of the methylene group that carry the open bond are not chlorine and that one of them at least is a fluorine;
GEA is a hydrocarbon group or an electroattractor group (i.e., the Hammett constant σₚ (sigma p) is >0, advantageously at least equal to 0.2, preferably inert, advantageously when p is equal to 1, an electroattractor group; and
p is a positive integer.

9. A compound according to claim 1, wherein R1 is hydrogen.

10. A compound according to claim 1, wherein R3 is hydrogen.

11. A compound according to claim 1, wherein R1 and R3 are hydrogen.

12. A compound according to claim 1, wherein R_{f} is a perfluoroalkyl of general formula CᵣF₂ₙ₊₁, where r is a positive integer from 1 to 10, advantageously from 1 to 5, preferably from 1 to 3.

13. A compound according to claim 1, wherein R_{f} is chosen from among the trifluoromethyl, pentafluoroethyl and heptafluoropropyl radicals.

14. A compound according to claim 1, wherein Ψ is an acyl.

15. A compound according to claim 1, wherein Ψ-O—H is an alkanoic acid with 1 to 8 carbon atoms.

16. A compound according to claim 1, wherein Ψ-O—H is an alkanoic acid with 2 to 5 carbon atoms.

17. A compound according to claim 1, wherein Ψ is an acyl such that the pKa of Ψ-O—H is at least equal to roughly 2, advantageously Ψ-O—H is an alkanoic acid with 1 to 8 carbon atoms, preferably from 2 to 5.

18. A process of synthesis of the compound of formula (1), wherein it entails bringing into contact a compound of formula (III): 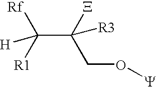
where [symbol] is a starting group that is chosen from among pseudohalogens and halogens, advantageously chlorine or bromine;
with a base that is chosen from among strong nitrogen-containing bases, of which the associated acid has a pKa that is at least equal to 12 and/or among anionic bases with the condition that when the base is a non-nitrogen-containing anionic base, the latter is in the presence of a solvent or a mixture of polar solvents.

19. A process according to claim 18, wherein the base is a non-nitrogen-containing anionic base and wherein said polar solvent has a solvent whose donor index is at least equal to 10.

20. A process according to claim 18, wherein the base is a non-nitrogen-containing anionic base and wherein said polar solvent has a solvent whose donor index is at least equal to 15.

21. A process according to claim 18, wherein the base is a non-nitrogen-containing anionic base and wherein said polar solvent has a solvent whose donor index is at least equal to 20.

22. A process according to claim 18, wherein the base is a non-nitrogen-containing anionic base and wherein said polar solvent is a water-miscible solvent in any proportion.

23. A process according to claim 18, wherein the base is a non-nitrogen-containing anionic base and wherein said polar solvent does not have an acid function.

24. A process according to claim 18, wherein said base is a non-nitrogen-containing anionic base, advantageously chosen from among salts, especially alkaline or alkaline-earth salts, of silylated amines and silyl amines.

25. A process according to claim 18, wherein said base is the anion of a silylamine chosen from among alkaline and alkaline-earth salts of HMDZ (hexamethyldisilazane).

26. A process according to claim 18, wherein said base is used in the presence of a polar, advantageously aprotic, solvent.

27. A process according to claim 18, wherein said base is a base that carries at least 2 trivalent nitrogens.

28. A process according to claim 18, wherein said base that carries at least 2 trivalent nitrogens is such that said 2 nitrogens are conjugated via at least one double bond.

29. A process according to claim 18, wherein said base that carries at least 2 trivalent nitrogens is such that said 2 trivalent nitrogens form a bond system comprising an imine conjugated with the doublet of an amine.

30. A process for making a heterocyclic compound substituted by a group R_{f} comprising the cyclocondensation of the compound of formula (I) with a co-substrate that carries 2 double bonds.

31. A process of claim 30, wherein said cyclocondensation is of type 3+2.

32. A process of claim 30, wherein the co-substrate is an organic compound that carries a pentavalent nitrogen, that itself carries 2 double bonds (including donor-acceptor type bonds), of which at least one double bond links said nitrogen to a carbon.
